(19) United States
(12) Patent Application Publication
(10) Pub. No.: US 2022/0033871 A1
(43) Pub. Date: Feb. 3, 2022

(54) PEPTIDE MARKERS FOR AUTHENTICATION OF EDIBLE BIRD'S NEST AND RELATED PRODUCTS

(71) Applicant: Hong Kong Baptist University, Hong Kong (CN)

(72) Inventors: Quanbin HAN, Hong Kong (CN); Lifeng LI, Hong Kong (CN); Wenjie WU, Hong Kong (CN)

(21) Appl. No.: 17/443,968

(22) Filed: Jul. 29, 2021

Related U.S. Application Data
(60) Provisional application No. 63/060,659, filed on Aug. 3, 2020.

(51) Int. Cl. C12P 21/06; G01N 30/88

(52) U.S. Cl. CPC C12P 21/06; G01N 2030/8831; G01N 30/88

(57) ABSTRACT

Provided herein is a method of authenticating using peptide markers found in edible bird's nest hydrolysate. The method can be used to authenticate edible bird's nest and related products and/or distinguish between white edible bird's nest and grass edible bird's nest.

### CROSS-REFERENCE TO RELATED APPLICATIONS

The present application claims priority to U.S. provisional patent application Ser. No. 63/060,659, filed on Aug. 3, 2020, and the disclosure of which is incorporated herein by reference in its entirety.

### TECHNICAL FIELD

The present disclosure relates to a series of peptide markers found in edible bird's nest (EBN) hydrolysate useful for authentication of EBN and related products. The present disclosure also provides a method to identify white EBN, grass EBN in raw material, and related products by using these peptide markers.

### BACKGROUND

EBN (Yan wo, in Chinese), created by the swiftlet, is a valuable food product from the Southeast Asia. In the market, EBN is classified into three types containing white, feather and grass nest. Traditionally, EBN is rich in protein and often served as a food tonic believed to have health benefits. However, with the increase in demand for EBN, there is an increase in news reporting imitation or adulterated EBN or selling a low quality EBN with a high price.

The number of documented and suspected cases of imitation or adulterated EBN with less expensive materials, such as agarose and snow fungus, which include polysaccharides, and gelatin and pork skins, which include polypeptides, has risen markedly over the past few years. The adulterants can have similar appearance and taste to EBN. Hence, it is difficult for people to distinguish imitation or adulterated EBN from authentic EBN by simple visual appearance or even taste. This is particularly true, when these imitation or adulterated EBN are used as instant EBN products.

The addition of adulterants may reduce the health benefits of EBN or even pose health risks. In addition, grass EBN is often used sold as white EBN due to lack of effective methods for identifying types of EBN. It is widely acknowledged that when comparing white EBN, grass EBN has poorer taste and is harder to soak to be expanded. Its price is thus several times lower than the white EBN in the market. Thus, the misusage is also considered as a violation of consumers' rights. These issues show the importance of authentication of EBN, otherwise the market is still in chaos and consumers' health and benefit will be harmed.

Traditional methods for authentication of EBN typically rely on the use sialic acid and protein as standard authentication biomarkers. However, sialic acid can also be found in other food, such as eggs, red meat or dairy products. For the protein analysis, real-time PCR (targeting gene of fibrinogen and NADH dehydrogenase) and two-dimensional gel electrophoresis were used in Chinese standard methods of authentication. In other studies, amino acid and peptide fingerprints were employed for EBN authentication. However, these approaches have low specificity, since other products may also have these genes and proteins. Moreover, the authentication of products containing mixtures of EBN and other materials is very challenging for current authentication methods due to markers that can interfere with the authentication.

Recently, efforts on the use of peptide markers for EBN authentication have involved shotgun proteomics combined with multiple reaction monitoring (MRM) analysis. This method is more specific, but with a series of steps for protein extraction, limited protein information was discovered. Additionally, the whole procedure is time-consuming and labor-intensive, thus not practicable for multiple batches of testing. More importantly, none of these approaches could distinguish white EBN from the grass EBN.

There thus exists a need to developed an improved method for EBN authentication that is simple and low-cost, high sensitivity and specificity, repeatable, and practical for commercial application.

### SUMMARY

Accordingly, it is an objective of the present disclosure to provide a method of authenticating of (EBN) and related products.

In a first aspect, provided herein is a method of identifying an edible bird's nest (EBN) in a sample suspected of comprising the EBN, the method comprising: providing a hydrolysate of the sample; analyzing the hydrolysate using a mass spectroscopy method; determining whether the hydrolysate comprises one or more peptide markers, wherein the one or more peptide markers have an observed mass to charge ratio (m/z) selected from the group consisting of: 277.6012-277.7012 (BNM201), 280.1454-280.2454 (BNM202), 280.6246-280.7246 (BNM203), 292.1148-292.2148 (BNM204), 294.7642-294.8642 (BNM205), 300.0959-300.1959 (BNM206), 305.1166-305.2166 (BNM207), 321.6531-321.7531 (BNM208), 373.7747-373.8747 (BNM209), 381.6542-381.7542 (BNM210), 391.6929-391.7929 (BNM211), 404.1486-404.2486 (BNM212), 410.6669-410.7669 (BNM213), 433.6506-433.7506 (BNM214), 441.6666-441.7666 (BNM215), 454.6518-454.7518 (BNM216), 468.6939-468.7939 (BNM217), 477.6830-477.7830 (BNM218), 498.7467-498.8467 (BNM219), 630.7600-630.8600 (BNM220), 711.3910-711.3910 (BNM221), 820.3134-820.4134 (BNM222), 844.3228-844.4228 (BNM223), 335.1730-335.2730 (BNM224), 417.6510-417.7510 (BNM225), and 447.6496-447.7496 (BNM226); and identifying based on the whether the hydrolysate comprises the one or more peptide markers if the sample comprises the EBN.

In certain embodiments, the method further comprises the step of hydrolyzing the sample thereby forming the hydrolysate of the sample.

In certain embodiments, the method further comprises of hydrolyzing the sample using a protease thereby forming the hydrolysate of the sample.

In certain embodiments, the protease is selected from the group consisting of trypsin, chymotrypsin, lysine protease, aspartic protease, pepsin, papain, proteinase K, calpain, and subtilisin.

In certain embodiments, the protease is trypsin.

In certain embodiments, the mass spectrometry method is tandem mass spectroscopy (MS/MS) and further comprises a liquid chromatography method.

The method of claim 1, wherein the mass spectrometry method comprises high-performance liquid chromatography (HPLC-MS/MS) or ultra-performance liquid chromatography (UPLC-MS/MS).

In certain embodiments, the step of determining whether the hydrolysate comprises one or more peptide markers further comprises determining whether each of the one or more peptide markers has a predicted liquid chromatography retention time, wherein the predicted liquid chromatography retention time is determined by measuring the retention time of standard samples, wherein each of the standard samples comprises one of the one or more peptide markers.

In certain embodiments, the one or more peptide markers having an observed m/z are selected from the group consisting of BNM212, BNM216, and BNM224.

In certain embodiments, the step of determining whether the hydrolysate comprises one or more peptide markers comprises determining whether the hydrolysate comprises 2 or 3 peptide markers selected from the group consisting of BNM212, BNM216, and BNM224.

In certain embodiments, the step of determining whether the hydrolysate comprises one or more peptide markers comprises determining whether the hydrolysate comprises 3 peptide markers selected from the group consisting of BNM212, BNM216, and BNM224.

In certain embodiments, the step of determining whether the hydrolysate comprises one or more peptide markers comprises determining whether the hydrolysate comprises 10 or more peptide markers, 15 or more peptide markers, 20 or more peptide markers, or 23 or more peptide markers.

In certain embodiments, the step of determining whether the hydrolysate comprises one or more peptide markers comprises determining whether the hydrolysate comprises 23 peptide markers selected from the group consisting of BNM201, BNM202, BNM203, BNM204, BNM205, BNM206, BNM207, BNM208, BNM209, BNM210, BNM211, BNM212, BNM213, BNM214, BNM215, BNM216, BNM217, BNM218, BNM219, BNM220, BNM221, BNM222, and BNM223; and optionally three peptide markers from the group consisting of BNM224, BNM225 and BNM226; and the step of identifying based on the whether the hydrolysate comprises the one or more peptide markers if the sample comprises EBN optionally comprises identifying if the sample comprises EBN, white EBN, or grass EBN.

In certain embodiments, the one or more peptide markers having an observed m/z are selected from the group consisting of BNM201, BNM202, BNM204, BNM205, BNM207, BNM208, BNM211, BNM213, BNM214, BNM215, BNM216, BNM217 and BNM221; and the step of identifying based on the whether the hydrolysate comprises the one or more peptide markers if the sample comprises EBN optionally comprises identifying if the sample comprises white EBN.

In certain embodiments, the step of determining whether the hydrolysate comprises one or more peptide markers comprises determining whether the hydrolysate comprises 2 or more peptide markers, 3 or more peptide markers, 4 or more peptide markers, 5 or more peptide markers, 6 or more peptide markers, 7 or more peptide markers, 8 or more peptide markers, 9 or more peptide markers, 10 or more peptide markers, 11 or more peptide markers, or 12 or more peptide markers from the group consisting of BNM201, BNM202, BNM204, BNM205, BNM207, BNM208, BNM211, BNM213, BNM214, BNM215, BNM216, BNM217 and BNM221.

In certain embodiments, the step of determining whether the hydrolysate comprises one or more peptide markers comprises determining whether the hydrolysate comprises thirteen peptide markers from the group consisting of BNM201, BNM202, BNM204, BNM205, BNM207, BNM208, BNM211, BNM213, BNM214, BNM215, BNM216, BNM217 and BNM221.

In certain embodiments, the one or more peptide markers having an observed m/z are selected from the group consisting of BNM224, BNM225 and BNM226; and the step of identifying based on the whether the hydrolysate comprises the one or more peptide markers if the sample comprises EBN optionally comprises identifying if the sample comprises grass EBN.

In certain embodiments, the step of determining whether the hydrolysate comprises one or more peptide markers comprises determining whether the hydrolysate comprises 2 or 3 peptide markers selected from the group consisting of BNM224, BNM225 and BNM226.

In certain embodiments, the method comprises: providing a trypsin hydrolysate of the sample; analyzing the trypsin hydrolysate using an UPLC-MS/MS method; determining whether the trypsin hydrolysate comprises peptide markers selected from the group consisting of: 3 peptide markers having an observed m/z are selected from the group consisting of BNM212, BNM216, and BNM224; 23 peptide markers having an observed m/z are selected from the group consisting of BNM201, BNM202, BNM203, BNM204, BNM205, BNM206, BNM207, BNM208, BNM209, BNM210, BNM211, BNM212, BNM213, BNM214, BNM215, BNM216, BNM217, BNM218, BNM219, BNM220, BNM221, BNM222, and BNM223; 13 peptide markers having an observed m/z are selected from the group consisting of BNM201, BNM202, BNM204, BNM205, BNM207, BNM208, BNM211, BNM213, BNM214, BNM215, BNM216, BNM217 and BNM221; and 3 peptide markers having an observed m/z are selected from the group consisting of BNM224, BNM225, and BNM226; and identifying based on the whether the trypsin hydrolysate comprises the peptide markers if the sample comprises EBN, white EBN, or grass EBN.

In certain embodiments, the step of determining whether the hydrolysate comprises one or more peptide markers further comprises determining whether each of the one or more peptide markers has a predicted ultra-performance liquid chromatography retention time, wherein the ultra-performance predicted liquid chromatography retention time is determined by measuring the retention time of standard samples, wherein each of the standard samples comprises one of the one or more peptide markers.

Peptides Production and Peptide Marker Selection for EBN Authentication

In accordance with a second aspect of the present disclosure, there is provided a series of peptide markers. These peptides were derived from EBN after direct trypsin digestion. These peptides were specific to EBN. This aspect comprises:

a. directly digesting EBN and other commonly used adulterants to peptides mixtures;

b. generating the peptide profiles; and

c. selecting the highly specific peptides as peptide markers.

In an embodiment of the second aspect wherein the step of digesting EBN is conducted by incubating with excess trypsin. The EBN is totally dried and powered. The powder without any pretreatment is directly dispersed in solution and mixed with trypsin. After digestion, no obvious insoluble substance should be observed.

In certain embodiments of the second aspect wherein other adulterants include agar, egg white, gelatin, cow milk, pork skin, tremella fungus, rice flour, starch and swim bladder.

In certain embodiments of the second aspect wherein the analysis of the generated peptides is performed on a C18 column detected by UPLC-ESI-qTOE-MS/MS.

In certain embodiments of the second aspect wherein the peptide marker is selected base on the difference in mass to charge ratio, MS/MS fragmentation and retention time. The peptide markers are confirmed by comparing multiple batches of EBN and adulterants. In certain embodiments, the selected peptide markers satisfy one or more of the following conditions: 1) has high specificity-is only presented in digested EBN instead of the mentioned adulterant; 2) is highly stable-existing in multiple batches of EBN.

Peptide Marker Selection for White and Grass EBN Differentiation

In accordance with a third aspect of the present disclosure, there is provided a series of peptide markers. These peptides were from white EBN and grass EBN after direct trypsin digestion. These peptides were specific to white EBN and grass EBN, respectively. This aspect comprises:

a. directly digesting white and grass EBN to peptides mixtures;

b. generating the peptide profiles;

c. selecting the highly specific peptides to white EBN and grass EBN by comparing with other adulterants, repetitively; and

d. selecting the peptide markers specific to white or grass EBN by comparing with each other.

In certain embodiments of the third aspect wherein the step of digesting white and grass EBN and generating peptide profiles are the same as the step involved in the second aspect.

In certain embodiments of the third aspect wherein the other adulterants include agar, egg white, gelatin, cow milk, pork skin, tremella fungus, rice flour, starch and swim bladder.

In certain embodiments of the third aspect wherein the peptide marker is selected base on the difference in mass to charge ratio, MS/MS fragmentation and retention time. The peptide markers are confirmed by comparing multiple batches of white and grass EBN. The selected peptide markers should satisfy the following conditions: 1) has high specificity-is only presented in digested white or grass EBN instead of the mentioned adulterant; 2) is highly stable-existing in multiple batches of white EBN or grass EBN.

Peptide Marker Determination in EBN Related Products

In accordance with a fourth aspect of the present disclosure, there is provided an application of EBN authentication, white and grass EBN identification in products.

a. detecting the peptide marker of EBN;

b. detecting the peptide marker of white EBN; and

c. detecting the peptide marker of grass EBN.

In certain embodiments of the fourth aspect wherein the step of detecting EBN, white EBN and grass EBN in products is applied in multiple batches of products collected from the market. One typical peptide marker could be used as an example for each case. The detection is conducted by mass spectroscopy analysis, such as by UPLC-ESI-Q-TOF-MS/MS analysis. The retention time of the peptide and mass to charge ratio of the marker can be used to identify and confirm the existence of the peptide markers.

### BRIEF DESCRIPTION OF THE DRAWINGS

The above and other objects and features of the present invention will become apparent from the following description of the invention, when taken in conjunction with the accompanying drawings, in which:

FIG. 1A shows the typical morphology of EBN including white, red and yellow nest (Yan Zhan), feather nest (Mao Yan), white strip (Yan Tiao), white pieces (Yan Bing), Broken white nest (Yan Sui), Grass nest or strip (Cao Yan) in the market.

FIG. 1B shows the typical morphology of Edible bird's related adulterants in the market.

FIG. 2A shows typical peptide profile-base peak chromatograms (BPC) of digested EBN sample. The peptides were separated on a column of ACQUITY UPLC BEH C18 (2.1 mm×100 mm, 1.7 μm).

FIG. 2B shows a typical peptide profile—BPC of digested Edible bird's related adulterant, agar. The peptides were separated on a column of ACQUITY UPLC BEH C18 (2.1 mm×100 mm, 1.7 μm).

FIG. 2C shows a typical peptide profile—BPC of digested Edible bird's related adulterant, egg white. The peptides were separated on a column of ACQUITY UPLC BEH C18 (2.1 mm×100 mm, 1.7 μm).

FIG. 2D shows a typical peptide profile—BPC of digested Edible bird's related adulterant, gelatin. The peptides were separated on a column of ACQUITY UPLC BEH C18 (2.1 mm×100 mm, 1.7 μm).

FIG. 2E shows a typical peptide profile—BPC of digested Edible bird's related adulterant, cow milk. The peptides were separated on a column of ACQUITY UPLC BEH C18 (2.1 mm×100 mm, 1.7 μm).

FIG. 2F shows a typical peptide profile—BPC of digested Edible bird's related adulterant, pork skin. The peptides were separated on a column of ACQUITY UPLC BEH C18 (2.1 mm×100 mm, 1.7 μm).

FIG. 2G shows a typical peptide profile—BPC of digested Edible bird's related adulterant, rice flour. The peptides were separated on a column of ACQUITY UPLC BEH C18 (2.1 mm×100 mm, 1.7 μm).

FIG. 2H shows a typical peptide profile—BPC of digested Edible bird's related adulterant, starch. The peptides were separated on a column of ACQUITY UPLC BEH C18 (2.1 mm×100 mm, 1.7 μm).

FIG. 2I shows a typical peptide profile—BPC of digested Edible bird's related adulterants, swim bladder. The peptides were separated on a column of ACQUITY UPLC BEH C18 (2.1 mm×100 mm, 1.7 μm).

FIG. 2J shows a typical peptide profile—BPC of digested Edible bird's related adulterant, tremella fungus. The peptides were separated on a column of ACQUITY UPLC BEH C18 (2.1 mm×100 mm, 1.7 μm).

FIG. 3A shows extracted ion chromatograms (EIC) of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM203.

FIG. 3B shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM206.

FIG. 3C shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM209.

FIG. 3D shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM210.

FIG. 3E shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM212.

FIG. 3F shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM218.

FIG. 3G shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM219.

FIG. 311 shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM220.

FIG. 31 shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM222.

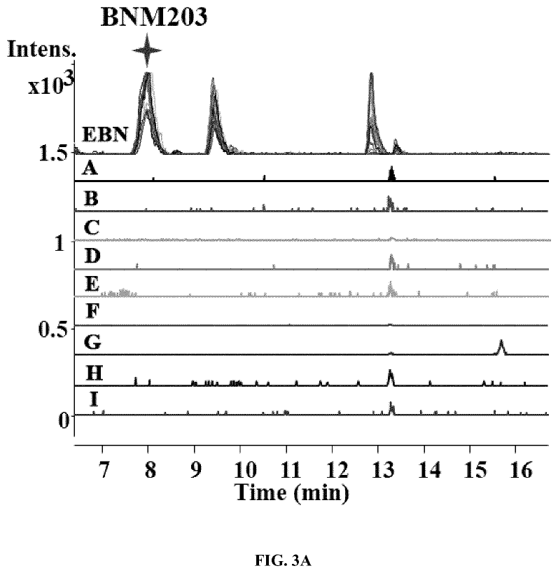

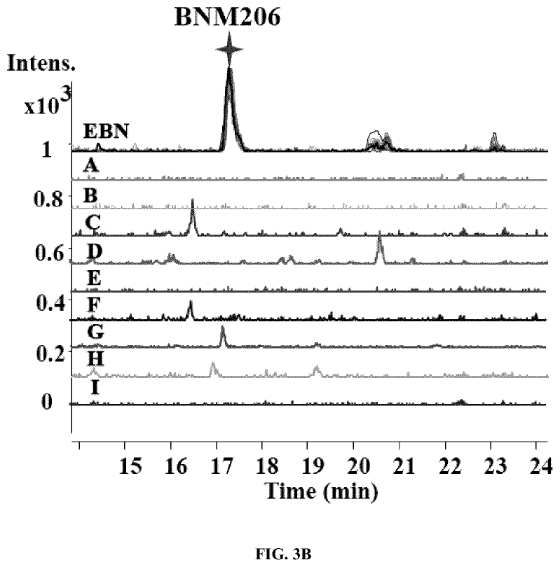

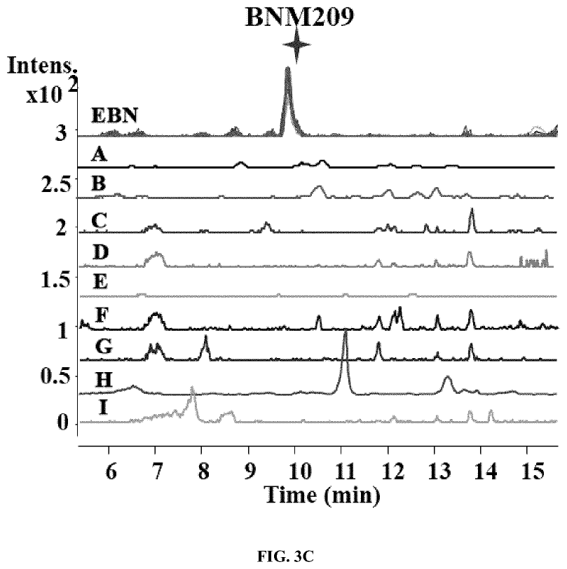

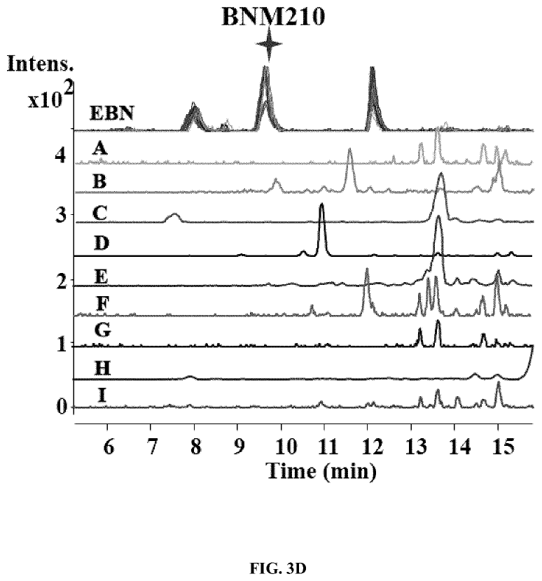

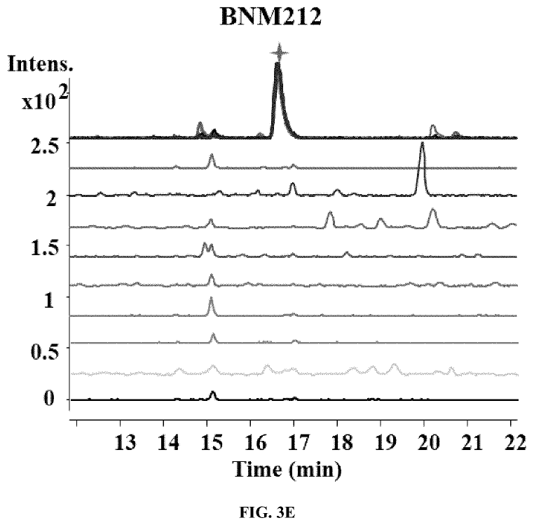

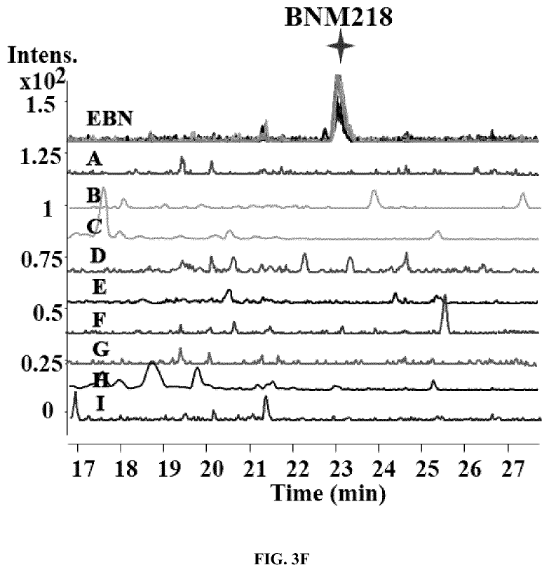

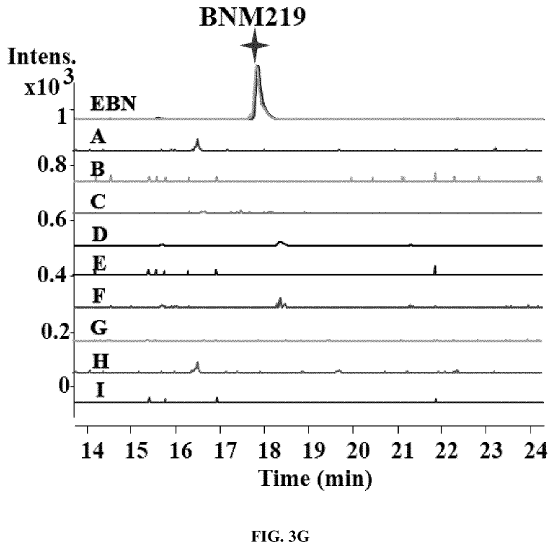

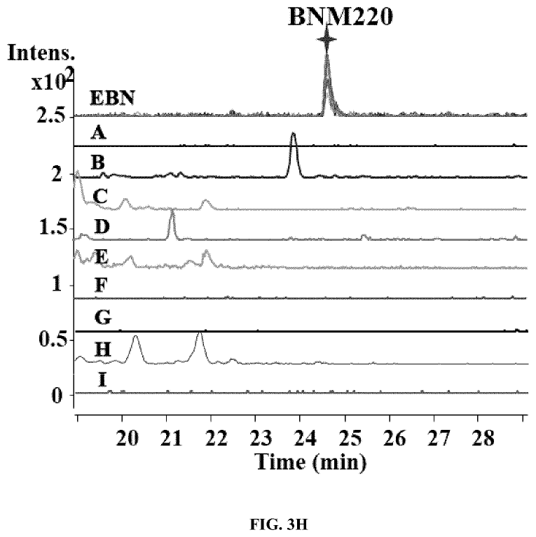

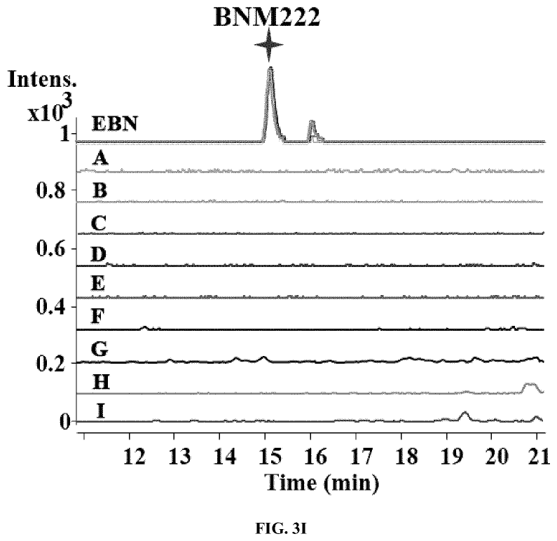

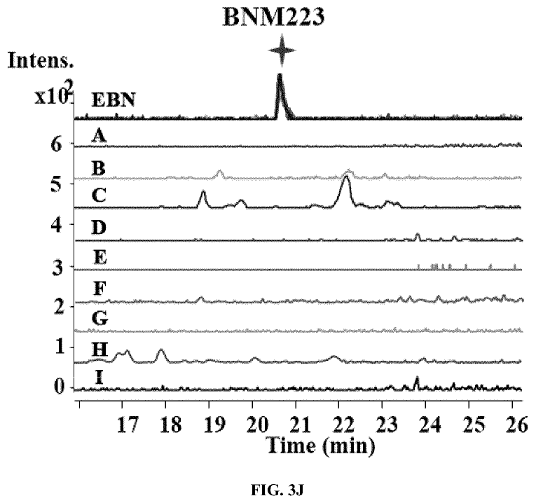

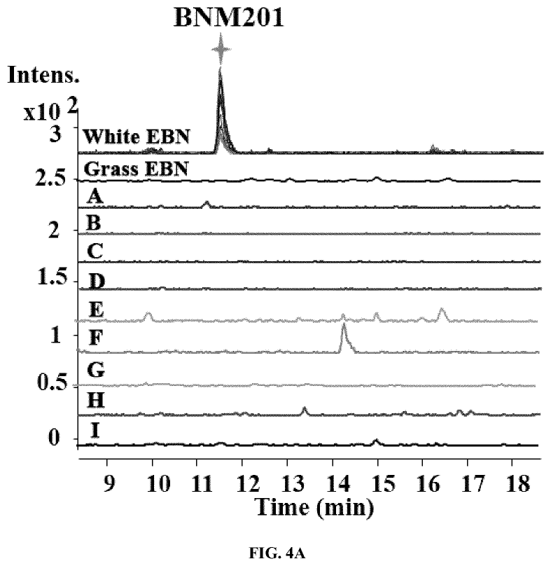

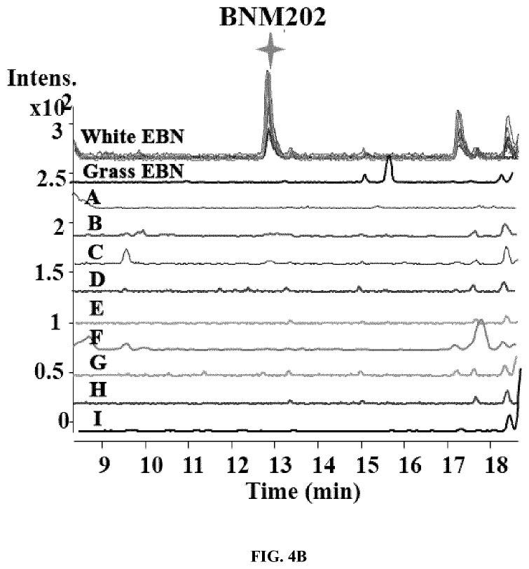

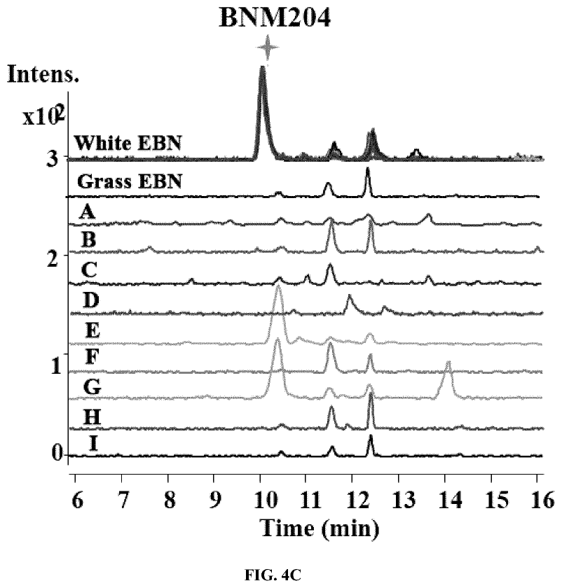

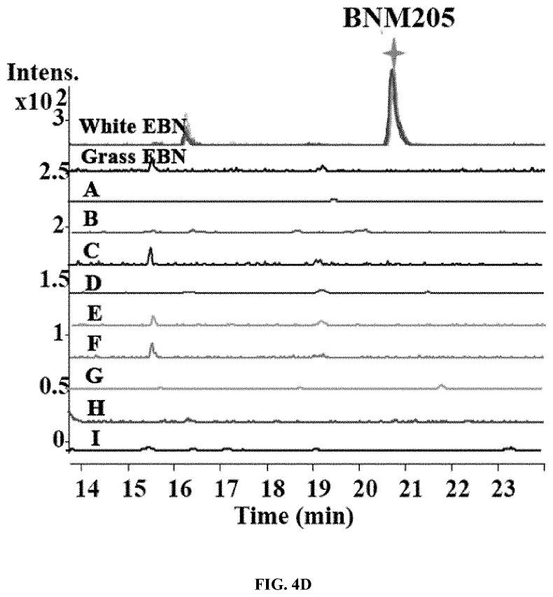

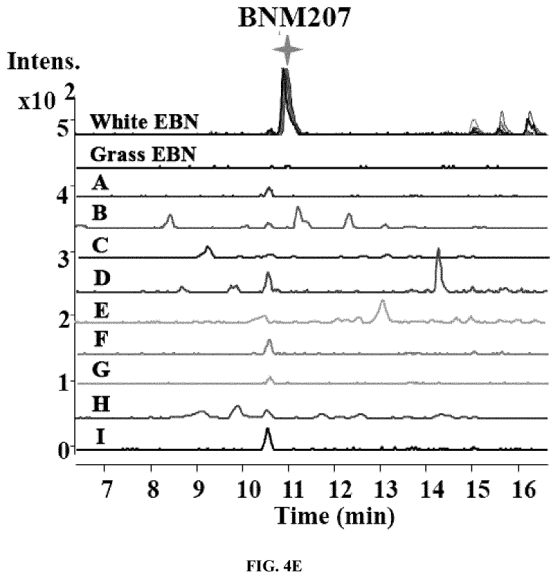

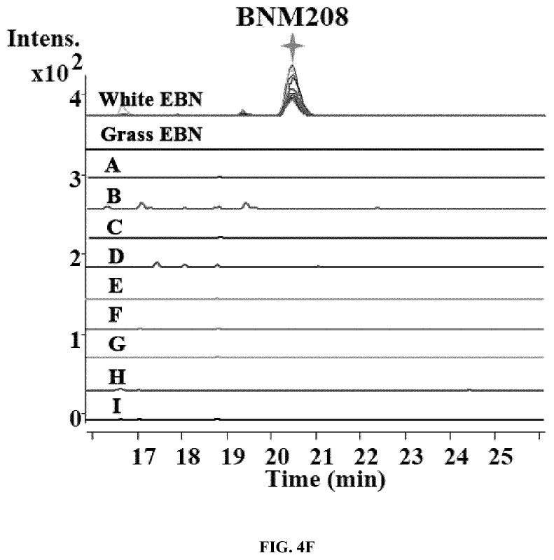

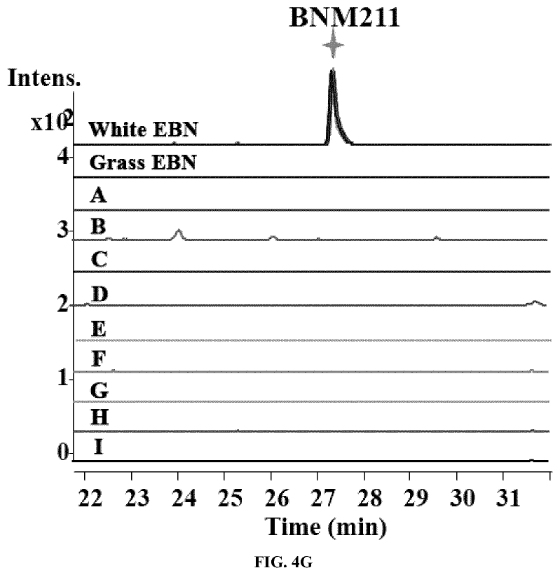

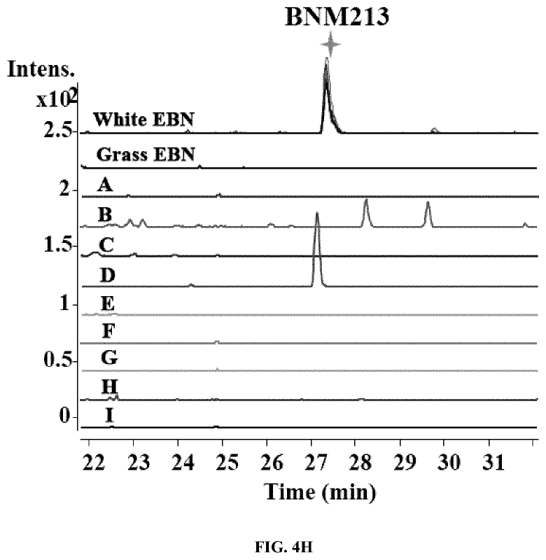

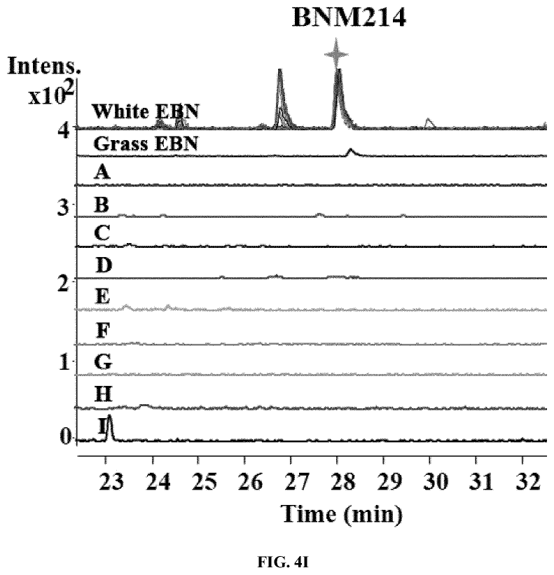

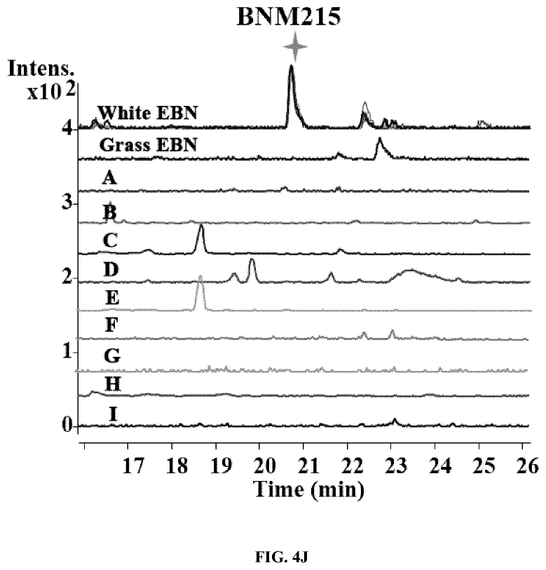

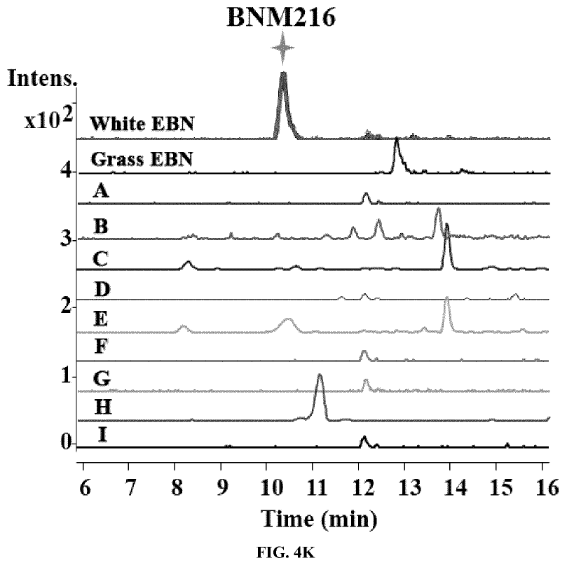

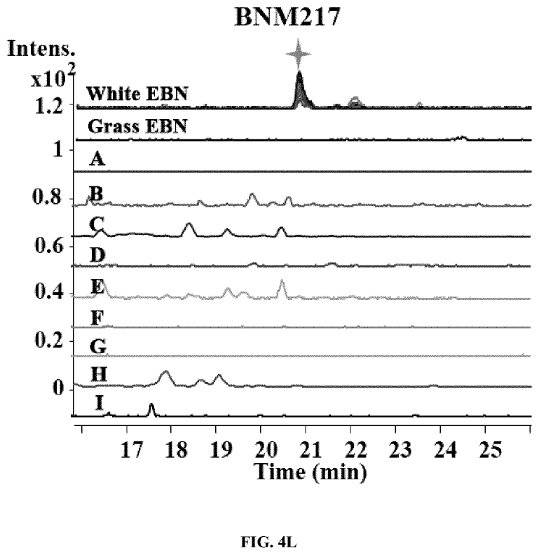

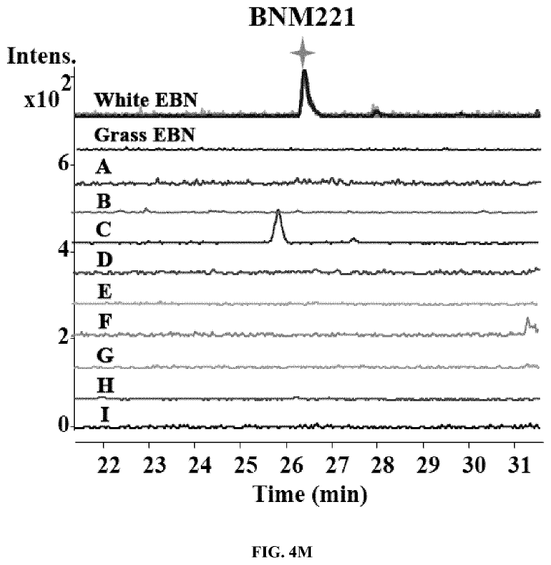

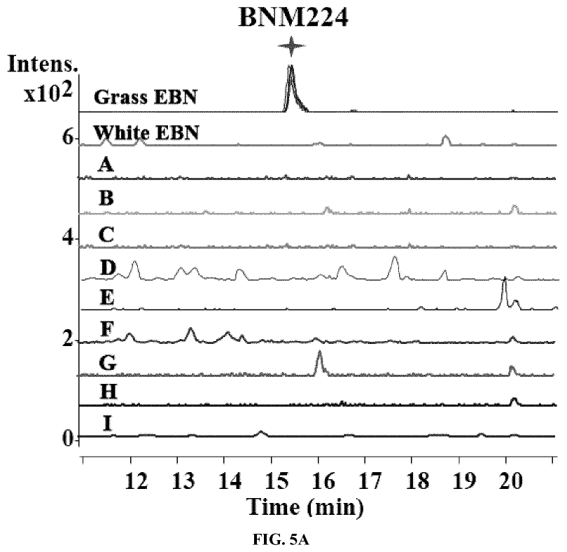

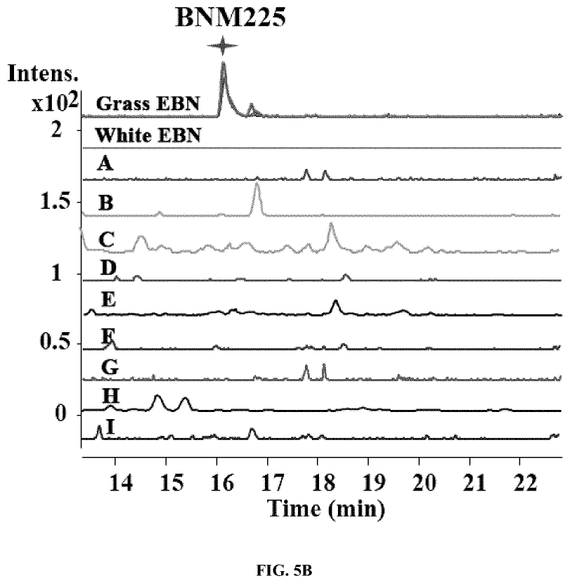

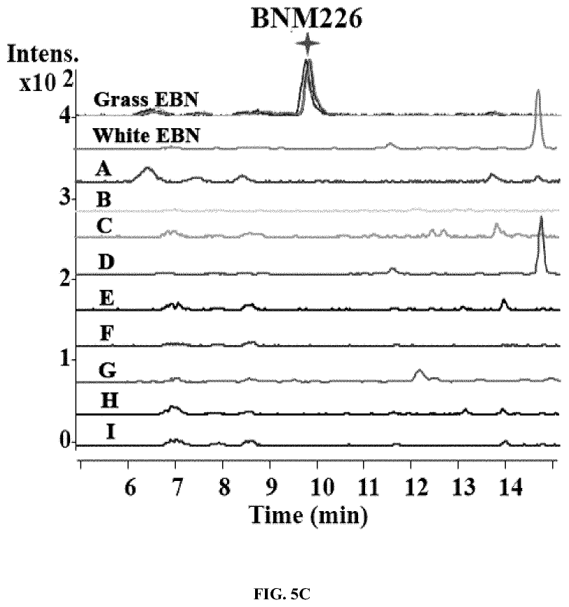

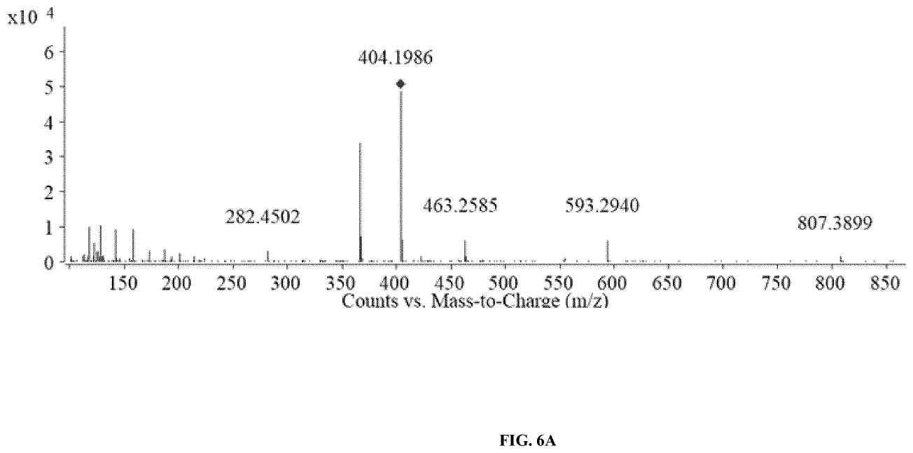

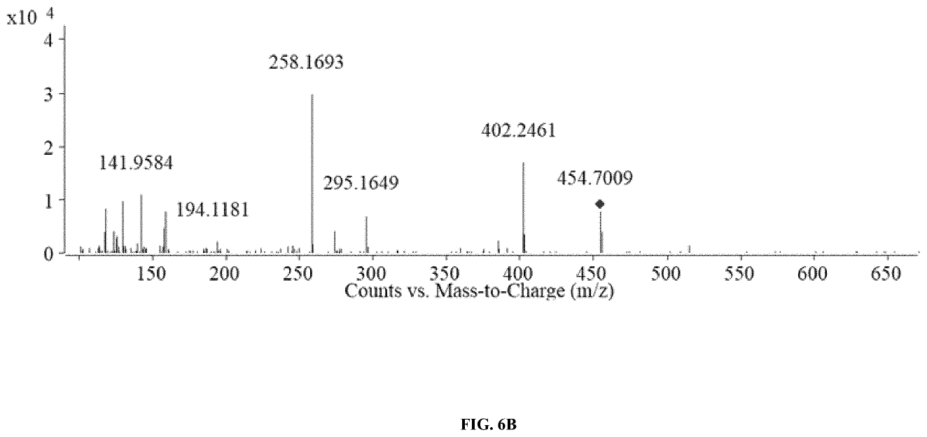

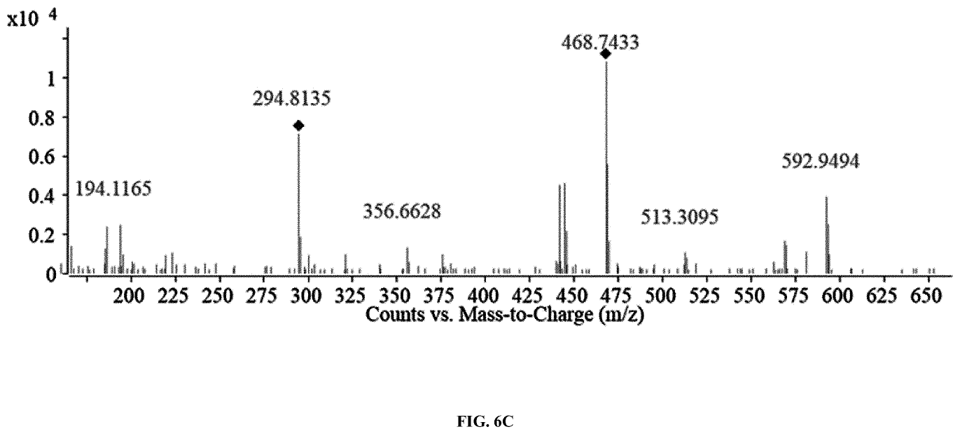

FIG. 3J shows EIC of selected peptide markers specific to EBN in typical batch of digested EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM223.

FIG. 4A shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM201.

FIG. 4B shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM202.

FIG. 4C shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM204.

FIG. 4D shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM205.

FIG. 4E shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM207.

FIG. 4F shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM208.

FIG. 4G shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM211.

FIG. 411 shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM213.

FIG. 41 shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM214.

FIG. 4J shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM215.

FIG. 4K shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM216.

FIG. 4L shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM217.

FIG. 4M shows EIC of selected peptide markers specific to white EBN in typical batch of digested white EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM221.

FIG. 5A shows EIC of selected peptide markers specific to grass EBN in typical batch of digested grass EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM224.

FIG. 5B shows EIC of selected peptide markers specific to grass EBN in typical batch of digested grass EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM225.

FIG. 5C shows EIC of selected peptide markers specific to grass EBN in typical batch of digested grass EBN (overlapped) and related adulterants. The adulterants include agar (A), egg white (B), gelatin (C), cow milk (D), pork skin (E), rice flour (F), starch (G), swim bladder (H) and tremella fungus (I). The peptide marker is BNM226.

FIG. 6A shows the LC-ESI-MS/MS spectra of ions corresponding to the identified peptide marker BNM212.

FIG. 6B shows the LC-ESI-MS/MS spectra of ions corresponding to the identified peptide marker BNM216.

FIG. 6C shows the LC-ESI-MS/MS spectra of ions corresponding to the identified peptide marker BNM217.

### DETAILED DESCRIPTION

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers but not the exclusion of any other integer or group of integers.

The term “sample” as used herein relates to a material or mixture of materials, typically, although not necessarily, in fluid form, but can also be in solid form, suspected of containing or containing EBN.

The present disclosure provides a method for identifying, authenticating, or distinguishing an EBN in a sample suspected of comprising the EBN or comprising the EBN, the method comprising: providing a hydrolysate of the sample; analyzing the hydrolysate using a mass spectroscopy method; determining whether the hydrolysate comprises one or more peptide markers, wherein the one or more peptide markers have an observed m/z selected from the group consisting of: 277.6012-277.7012 (BNM201), 280.1454-280.2454 (BNM202), 280.6246-280.7246 (BNM203), 292.1148-292.2148 (BNM204), 294.7642-294.8642 (BNM205), 300.0959-300.1959 (BNM206), 305.1166-305.2166 (BNM207), 321.6531-321.7531 (BNM208), 373.7747-373.8747 (BNM209), 381.6542-381.7542 (BNM210), 391.6929-391.7929 (BNM211), 404.1486-404.2486 (BNM212), 410.6669-410.7669 (BNM213), 433.6506-433.7506 (BNM214), 441.6666-441.7666 (BNM215), 454.6518-454.7518 (BNM216), 468.6939-468.7939 (BNM217), 477.6830-477.7830 (BNM218), 498.7467-498.8467 (BNM219), 630.7600-630.8600 (BNM220), 711.3910-711.3910 (BNM221), 820.3134-820.4134 (BNM222), 844.3228-844.4228 (BNM223), 335.1730-335.2730 (BNM224), 417.6510-417.7510 (BNM225), and 447.6496-447.7496 (BNM226); and identifying based on the whether the hydrolysate comprises the one or more peptide markers if the sample comprises the EBN.

The sample can be derived from a variety of sources, such as from processed or unprocessed EBN, food stuffs, herbal medicine, and extracts thereof.

The sample can optionally be treated before and/or after the hydrolysis step in order to, e.g., improve sample handling or sample properties or simplify mass spectroscopy analysis. The sample or the hydrolysate can be optionally treated by washing, extraction, reduction, alkylation, deglycosylation, and the like. Advantageously, however, the methods described herein do not require pre-treatment, such as by deglycosylation, reduction, and/or and alkylation steps in order to achieve EBN authentication with high specificity and sensitivity. Thus, in certain embodiments, the method described herein does not further comprise one or more pretreatment steps selected from the group consisting of deglycosylation, alkylation, and reduction.

The sample comprising the EBN can then be hydrolysed thereby forming the hydrolysate of the sample. Hydrolysis refers to the breakdown of proteins or polypeptides into shorter polypeptides, and oligopeptides and possibly, to some extent, individual amino acids by cleavage of one or more peptide bonds joining the constituent amino acids.

The method for hydrolysing the sample is not particularly limited. Any method for hydrolysing proteins or polypeptides known to those of ordinary skill in the art can be employed in the methods described herein. In certain embodiments, the hydrolysis of the sample is accomplished using a Brønsted acid, a Brønsted base, or a Lewis Acid catalysed aqueous hydrolysis or using an enzyme, such as a protease.

The protease can have broad specificity, so that all proteins and/or polypeptides in the sample are hydrolysed. Alternatively a mixture of specific or non-specific proteases may be used, to provide broader specificity.

In certain embodiments, the protease is selected from the group consisting of trypsin, chymotrypsin, pepsin, papain, proteinase K, calpain, subtilisin, and mixtures thereof. In certain embodiments, the protease is trypsin.

The appropriate conditions for the hydrolysis of the sample will vary with the protease used. The optimal pH, temperature, and digestion time can be a function of the protease utilized for the hydrolysis of the sample and changing the protease will change these and potentially other parameters. The selection of the appropriate protease and the hydrolysis conditions is well within the skill of a person of ordinary skill in the art.

The hydrolysate of the sample can comprise a mixture of intact proteins or polypeptides, shorter polypeptides, and oligopeptides and component amino acids, which are produced by hydrolysis. Such mixture can be analysed using a mass spectrometry methods to determine whether the sample comprises one or more peptide markers useful for identifying EBN in the sample.

Mass spectrometry is performed using a mass spectrometer comprising an ion source for ionizing the sample and creating charged molecules and/or charged fragments for further analysis. The ionization of the sample can be performed by electron ionization, chemical ionization, electrospray ionization (ESI), photon ionization, atmospheric pressure chemical ionization (APCI), photoionization, atmospheric pressure photoionization (APPI), fast atom bombardment (FAB), liquid secondary ionization (LSI), matrix assisted laser desorption ionization (MALDI), field ionization, field desorption, thermospray/plasmaspray ionization, surface enhanced laser desorption ionization (SELDI), inductively coupled plasma (ICP) and particle beam ionization. The person of ordinary skill in the art will understand that the choice of ionization method can be determined based on the properties of the analyte(s) being measured, type of sample, detector type, the choice of positive versus negative mode, etc. The ionizer can operate in positive or negative ion mode. In certain embodiments, the ionization of the sample is accomplished using ESI.

Once the sample has been ionized, the positively charged or negatively charged ions thereby created may be analysed to determine an m/z ratio. Exemplary analysers for determining m/z ratios include, but are not limited to, quadrupole analysers, ion traps analysers, and time-of-flight (TOF) analysers. In certain embodiments, the analyser is a tandem mass spectrometers (MS) selected from a triple quadrupole MS and 2 dual-focusing; and hybrid MS selected from the group consisting of quadrupole TOF (Q-TOF), ion trap TOF (IT-TOF), quadrupole ion trap (Q-IT), quadrupole-cyclotron-resonance (Q-ICR), ion trap ion-cyclotron-resonance (IT-ICR), ion trap orbitrap (IT-orbitrap), 2 TOF (TOF-TOF), and multistage MS (MSⁿ). The ions may be detected using several detection modes. For example, selected ions may be detected, i.e. using a selective ion monitoring mode (SIM), or alternatively, ions may be detected using a scanning mode, e.g., multiple reaction monitoring (MRM) or selected reaction monitoring (SRM). In certain embodiments, the m/z ratio is determined using a Q-TOF analyser.

In certain embodiments, the mass spectrometry further comprises liquid chromatograph prior to the step of analysing the hydrolysate sample by a mass spectrometry method. Liquid chromatography is a process of selective at least partial obstruction of one or more components of a fluid solution (mobile phase) as the mobile phase passes through a column of a substance, through capillary passageways, or through a single contiguous column of solid support, such as monolithic column. The at least partial obstruction results from the distribution of the components of the mixture between the stationary phase and mobile phase, as this mobile phase moves relative to the stationary phase(s). Examples of liquid chromatography include HPLC, UPLC [also known as ultrahigh performance liquid chromatograph (UHPLC)], and reverse phase liquid chromatography (RPLC). In certain embodiments, the mass spectrometry method further comprises UPLC, such as HPLC-MS, UPLC-MS, UPLC-MS/MS. In the examples below the mass spectrometry method comprises reverse phase UPLC-ESI-qTOE-MS/MS using a C18 column.

The m/z data generated as a result of the mass spectrometry analysis of the hydrolysate sample can be then be examined to determine whether the hydrolysate samples comprises ions that correspond with one or more peptide markers having an observed m/z that are useful for identifying, authenticating, and/or distinguishing EBN. In certain embodiments, the one or more peptide markers have an observed m/z selected from the group consisting of BNM201, BNM202, BNM203, BNM204, BNM205, BNM206, BNM207, BNM208, BNM209, BNM210, BNM211, BNM212, BNM213, BNM214, BNM215, BNM216, BNM217, BNM218, BNM219, BNM220, BNM221, BNM222, BNM223, BNM224, BNM225, and BNM226.

In certain embodiments, the step of determining whether the hydrolysate comprises one or more peptide markers comprises determining whether the hydrolysate comprises 2 or more peptide markers, 3 or more peptide markers, 4 or more peptide markers, 5 or more peptide markers, 6 or more peptide markers, 7 or more peptide markers, 8 or more peptide markers, 9 or more peptide markers, 10 or more peptide markers, 11 or more peptide markers, 12 or more peptide markers, 13 or more peptide markers, 14 or more peptide markers, 15 or more peptide markers, 16 or more peptide markers, 17 or more peptide markers, 18 or more peptide markers, 19 or more peptide markers, 20 or more peptide markers, 21 or more peptide markers, 22 or more peptide markers, 23 or more peptide markers, 24 or more peptide markers, 25 or more peptide markers; or 26 peptide markers.

The presence of one or more peptide markers have an observed m/z of BNM201-BNM226 can be used to identify EBN in the sample, authenticate EBN in the sample, or distinguish white EBN from grass EBN.

In certain embodiments, the presence of 1, 2, or 3 peptides markers in the mass spectrometry results having an observed m/z selected from the group consisting of BNM212, BNM216, and BNM224 is used to identify EBN in the sample or authenticate EBN in the sample.

In certain embodiments, the presence of 2 or more peptide markers, 3 or more peptide markers, 4 or more peptide markers, 5 or more peptide markers, 6 or more peptide markers, 7 or more peptide markers, 8 or more peptide markers, 9 or more peptide markers, 10 or more peptide markers, 11 or more peptide markers, 12 or more peptide markers, or 13 peptide markers in the mass spectrometry results having an observed m/z selected from the group consisting BNM201, BNM202, BNM204, BNM205, BNM207, BNM208, BNM211, BNM213, BNM214, BNM215, BNM216, BNM217 and BNM221 is used to identify white EBN in the sample or authenticate white EBN in the sample. In certain embodiments, the presence of 2 or more peptide markers, 3 or more peptide markers, 4 or more peptide markers, 5 or more peptide markers, 6 or more peptide markers, 7 or more peptide markers, 8 or more peptide markers, 9 or more peptide markers, 10 or more peptide markers, 11 or more peptide markers, 12 or more peptide markers, or 13 peptide markers in the mass spectrometry results having an observed m/z selected from the group consisting BNM201, BNM202, BNM204, BNM205, BNM207, BNM208, BNM211, BNM213, BNM214, BNM215, BNM216, BNM217 and BNM221; and the absence of, 2, or 3 peptide markers in the mass spectrometry results having an observed m/z selected from the group consisting BNM224, BNM225 and BNM226 is used to identify white EBN in the sample, authenticate white EBN in the sample, or distinguish between white EBN and grass EBN.

In certain embodiments, the presence of 1, 2, or 3 peptide markers in the mass spectrometry results having an observed m/z selected from the group consisting BNM224, BNM225 and BNM226 is used to identify grass EBN in the sample, authenticate grass EBN in the sample, or distinguish between grass EBN and white EBN.

The methods described herein can be used to determine if a sample suspected of comprising EBN comprises one or more adulterants or if the EBN is imitation EBN prepared from one or more adulterants. Exemplary adulterants include, but are not limited to, egg white, gelatin, cow milk, pork skin, tremella fungus, rice flour, starch, swim bladder, and the like.

In instances in which the mass spectrometry method further comprises liquid chromatography, the step of determining whether the hydrolysate comprises one or more peptide markers can further comprise determining whether each of the one or more peptide markers has a predicted liquid chromatography retention time, wherein the predicted liquid chromatography retention time is determined by measuring the retention time of standard samples, wherein each of the standard samples comprises one of the one or more peptide markers. The retention time of standard samples is preferably measured under substantially similar parameters as the hydrolysate sample. Such parameters can include, but are not limited to, column/solid media type, mobile phase solvent(s), mobile phase flow rate, pressure, temperature, and the like. The selection of liquid chromatography parameters is well within the skill of a person of ordinary skill in the art.

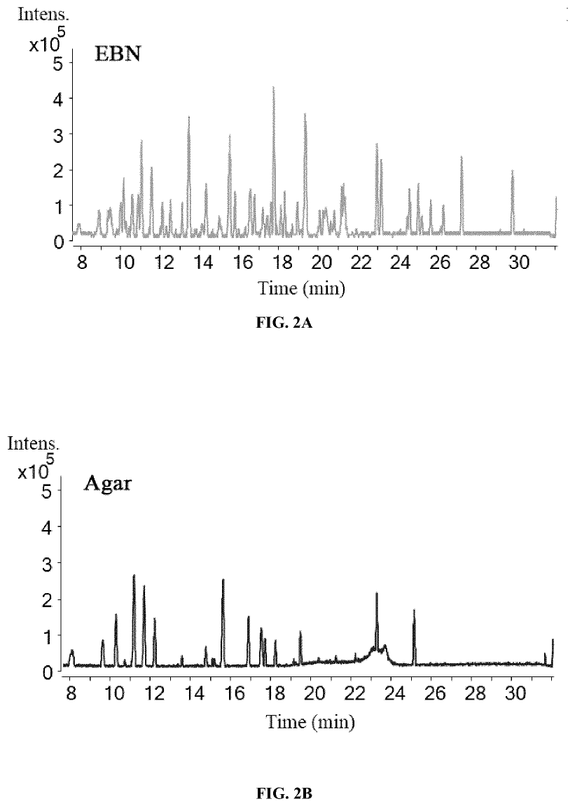

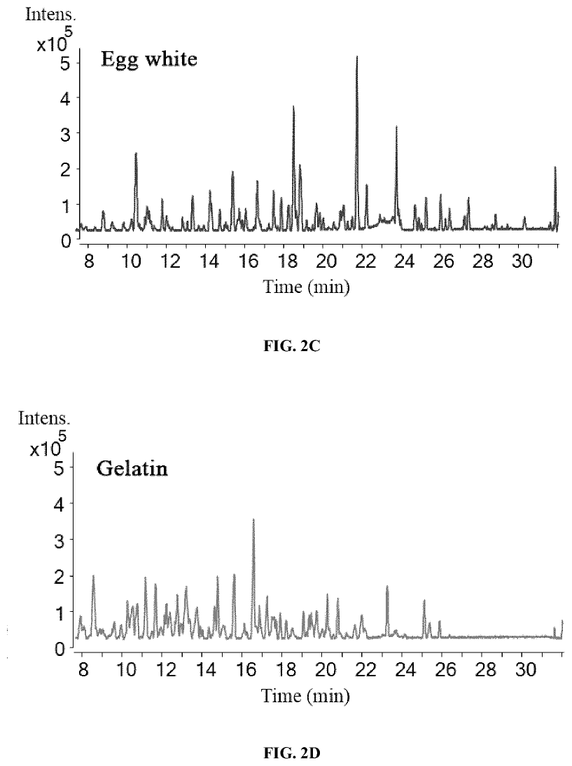

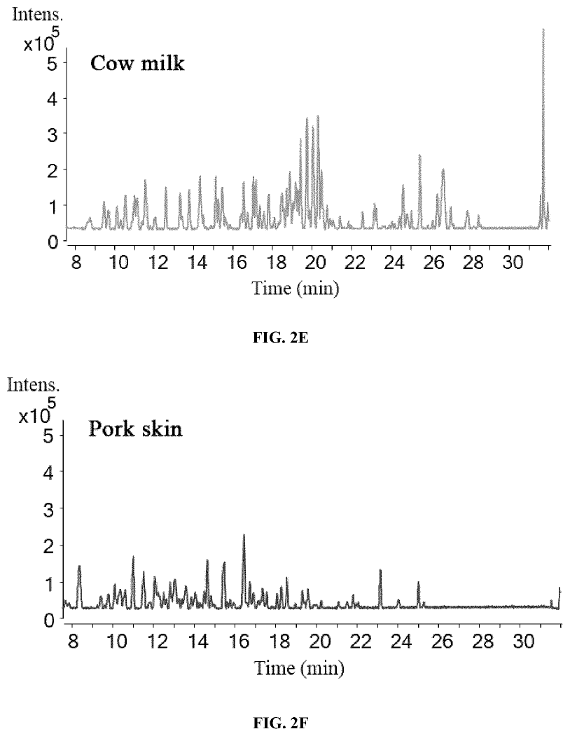

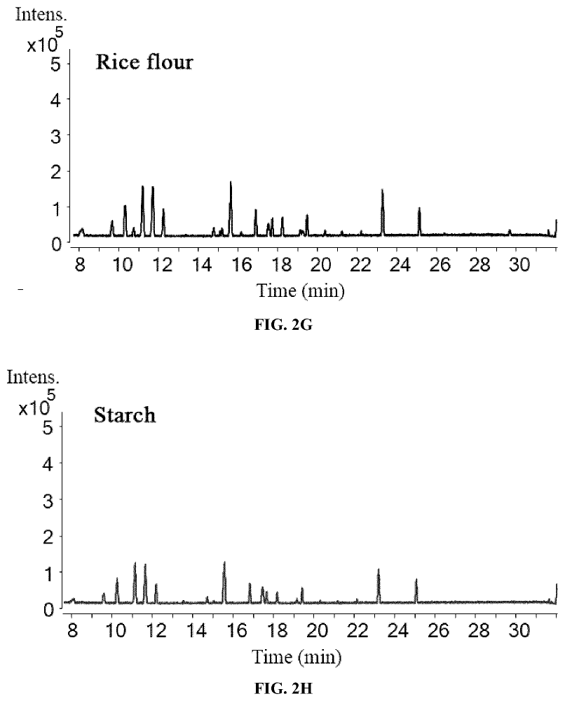

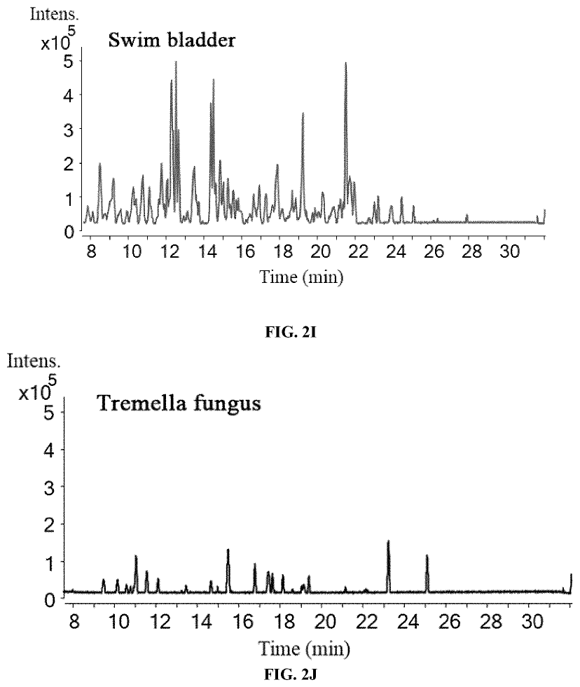

UPLC-MS analysis of the digested EBN and adulterants: As shown in FIG. 2A-2J, the peptide profile of the EBN (FIG. 2A) are quite similar with some adulterants, especially egg white (FIG. 2C). This might result from the high protein homology. Therefore, it is difficult to distinguish EBN and many common adulterants by visual observation of their chromatograms. The specific peptide markers were selected in further analysis.

Selection of the peptide markers specific to EBN: To discover the specific peptide markers, shotgun proteomics combined with multiple reaction monitoring (MRM) analysis was always employed. However, it will have some limitations when applied to EBN. For example, as most proteins in EBN are glycosylated with 0 and/or N-glycans, the modification increases the difficulty of protein separation, analysis and identification. Thus, complex pretreatment including glycan removal can be required. Simultaneously, reduction and alkylation steps may also be involved to provide additional protein information. These pretreatments will be more time-consuming and not practicable for commercial testing. Therefore, to make the authentication of EBN simpler and easy-to-operate, the methods described herein involve hydrolysis of EBN, e.g., by direct trypsin digestion, and comparison of the hydrolysates. Following the peptide marker selection criteria, comparing to related adulterants, there were totally 10 peptides found to be specific to EBN including both white and grass EBN as shown in Table 1. The EIC was described as shown in FIG. 3A-3G Authentication using one or more of these peptide markers can provide EBN authentication with high specificity and sensitivity. And these peptide markers were consistently present in the multiple batches of EBN (overlapped chromatograms) samples (n=70 including 60 batches of white EBN and 10 batches of grass EBN). Thus, these peptide markers are satisfactory as authenticating peptide markers.

TABLE 1
Selection of peptide markers of EBN based on retention time, mass-to-charge
ratio, specificity and consistency.

| | | | | | | | | EBN |
|---|---|---|---|---|---|---|---|---|
| | Observed | RT | | | | | common | peptide |
| No. | m/z | (min) | EBN | Adulterants^{a} | Specificity | Peptide | peaks^{b} | marker |
| 1 | 231.1710 | 10.67 | +^{c} | + | −^{c} | /^{c} | / | / |
| 2 | 237.1235 | 9.04 | + | + | − | / | / | / |
| 3 | 258.1691 | 10.24 | + | + | − | / | / | / |
| 4 | 258.6615 | 12.59 | + | − | − | / | / | / |
| 5 | 265.1557 | 13.54 | + | + | − | / | / | / |
| 6 | 272.1722 | 9.51 | + | + | − | / | / | / |
| 7 | 277.6512 | 11.49 | + | − | + | + | − | − |
| 8 | 280.1954 | 12.86 | + | − | + | + | − | − |
| 9 | 280.6746 | 7.97 | + | − | + | + | + | BNM203 |
| 10 | 288.2036 | 12.59 | + | − | + | − | / | / |
| 11 | 292.1648 | 10.05 | + | − | + | + | − | − |
| 12 | 294.8142 | 20.7 | + | − | + | + | − | − |
| 13 | 300.1459 | 17.25 | + | − | + | + | + | BNM206 |
| 14 | 305.1666 | 10.99 | + | − | + | + | − | − |
| 15 | 321.7031 | 20.45 | + | − | + | + | − | − |
| 16 | 336.1926 | 21.4 | + | − | + | − | / | / |
| 17 | 339.1850 | 10.99 | + | + | − | / | / | / |
| 18 | 344.2550 | 21.32 | + | + | − | / | / | / |
| 19 | 346.2350 | 19.48 | + | − | − | / | / | / |
| 20 | 347.6992 | 11.63 | + | + | − | / | / | / |
| 21 | 350.1712 | 15.09 | + | − | − | / | / | / |
| 22 | 350.2074 | 21.07 | + | − | − | / | / | / |
| 23 | 364.2235 | 19.48 | + | − | + | − | / | / |
| 24 | 366.2030 | 16.66 | + | − | − | / | / | / |
| 25 | 373.8247 | 9.82 | + | − | + | + | + | BNM209 |
| 26 | 375.2250 | 7.96 | + | + | − | / | / | / |
| 27 | 381.7042 | 9.61 | + | − | + | + | + | BNM210 |
| 28 | 382.1620 | 12.01 | + | − | − | / | / | / |
| 29 | 389.2400 | 11.09 | + | + | − | / | / | / |
| 30 | 391.7429 | 27.35 | + | − | + | + | − | − |
| 31 | 402.2467 | 10.24 | + | + | − | / | / | / |
| 32 | 404.1986 | 16.65 | + | − | + | + | + | BNM212 |
| 33 | 410.7169 | 27.35 | + | − | + | + | − | − |
| 34 | 433.7006 | 28.04 | + | − | + | + | − | − |
| 35 | 441.7166 | 20.65 | + | − | + | + | − | − |
| 36 | 454.7018 | 10.24 | + | − | + | + | − | − |
| 37 | 467.2138 | 10.38 | + | − | − | / | / | / |
| 38 | 468.7439 | 20.78 | + | + | + | + | − | − |
| 39 | 472.3140 | 25.78 | + | + | + | + | − | − |
| 40 | 477.7330 | 23.14 | + | + | + | + | + | BNM218 |
| 41 | 479.2879 | 24.73 | + | + | + | − | / | / |
| 42 | 491.3200 | 29.94 | + | + | + | − | / | / |
| 43 | 494.2616 | 14.44 | + | + | + | − | / | / |
| 44 | 498.7967 | 17.87 | + | + | + | + | + | BNM219 |
| 45 | 507.2456 | 18.39 | + | + | + | − | / | / |
| 46 | 509.2615 | 15.79 | + | + | + | − | / | / |
| 47 | 525.7603 | 22.46 | + | − | − | / | / | / |
| 48 | 548.2486 | 10.07 | + | − | − | / | / | / |
| 49 | 582.2528 | 11.01 | + | + | + | − | / | / |
| 50 | 629.3627 | 12.56 | + | + | + | − | / | / |
| 51 | 630.8100 | 24.56 | + | + | + | + | + | BNM220 |
| 52 | 711.4410 | 26.43 | + | + | + | + | − | − |
| 53 | 804.3734 | 16.24 | + | + | + | − | / | / |
| 54 | 820.3634 | 15.13 | + | + | + | + | + | BNM222 |
| 55 | 844.3728 | 20.63 | + | + | + | + | + | BNM223 |

Note:
^{a}the adulterants include egg white, gelatin, cow milk, pork skin, tremella fungus, rice flour, starch and swim bladder.
^{b}Common peaks are peptide markers which were consistently present in 70 batches of EBN. Both white (n = 60) and grass EBN (n = 10) have these peptide markers.
^{c}“+” is the positive result. “−” is the negative result. “/” is result that is not applicable.

Selection of the peptide markers to distinguish white EBN from grass EBN: Based on the criteria of peptide marker selection, 13 peptide markers were found to be specific to white EBN. While there were three peptide markers that could be used for grass EBN identification. The information is summarized in Table 2. The FIG. 4A-4M and FIG. 5A-5C show the high specificity of these peptide markers. Using these peptide markers, white EBN and grass EBN could be well-discriminated.

TABLE 2
Selection of peptide markers of white and grass EBN based on retention time,
mass-to-charge ratio, specificity and consistency.

| | | | | | | | White | Grass |
|---|---|---|---|---|---|---|---|---|
| | | | | | | | EBN | EBN |
| Peak | Observed | RT | White | Grass | | Specific | Peptide | peptide |
| No. | m/z | (min) | EBN^{b} | EBN^{b} | Adulterants ^{a} | peptide ^{d} | marker | marker |
| 7 | 277.6512 | 11.49 | +^{c} | −^{c} | − | + | BNM201 | − |
| 8 | 280.1954 | 12.86 | + | − | − | + | BNM202 | − |
| 9 | 280.6746 | 7.97 | + | + | − | + | − | − |
| 11 | 292.1648 | 10.05 | + | − | − | + | BNM204 | − |
| 12 | 294.8142 | 20.7 | + | − | − | + | BNM205 | − |
| 13 | 300.1459 | 17.25 | + | + | − | + | − | − |
| 14 | 305.1666 | 10.99 | + | − | − | + | BNM207 | − |
| 15 | 321.7031 | 20.45 | + | − | − | + | BNM208 | − |
| 25 | 373.8247 | 9.82 | + | + | − | + | − | − |
| 27 | 381.7042 | 9.61 | + | + | − | + | − | − |
| 30 | 391.7429 | 27.35 | + | − | − | + | BNM211 | − |
| 32 | 404.1986 | 16.65 | + | + | − | + | − | − |
| 33 | 410.7169 | 27.35 | + | − | − | + | BNM213 | − |
| 34 | 433.7006 | 28.04 | + | − | − | + | BNM214 | − |
| 35 | 441.7166 | 20.65 | + | − | − | + | BNM215 | − |
| 36 | 454.7018 | 10.24 | + | − | − | + | BNM216 | − |
| 38 | 468.7439 | 20.78 | + | − | − | + | BNM217 | − |
| 40 | 477.7330 | 23.14 | + | + | − | + | − | − |
| 44 | 498.7967 | 17.87 | + | + | − | + | − | − |
| 51 | 630.8100 | 24.56 | + | + | − | + | − | − |
| 52 | 711.4410 | 26.43 | + | − | − | + | BNM221 | − |
| 54 | 820.3634 | 15.13 | + | + | − | + | − | − |
| 55 | 844.3728 | 20.63 | + | + | − | + | − | − |
| 56 | 335.2230 | 15.39 | − | + | − | + | − | BNM224 |
| 57 | 417.7010 | 16.15 | − | + | − | + | − | BNM225 |
| 58 | 447.6996 | 9.81 | − | + | − | + | − | BNM226 |

Note:
^{a} the adulterants include egg white, gelatin, cow milk, pork skin, tremella fungus, rice flour, starch and swim bladder.
^{b}60 batches of white EBN and 10 batches of grass EBN were used.
^{c}“+” is the positive result. “−” is the negative result.
^{d} The peptide marker selected should be peptides and show high specificity comparing with the adulterants.

Peptide marker identification by searching database: As shown in Table 3, there were 26 peptide markers in total that could be used to identify EBN, white EBN and grass EBN. To identify the sequence of these peptide marker candidates, MS/MS data was used for searching the peptide databases. As shown in Table 4 and FIG. 6A 6C, three peptide markers were identified. The sequence was NTLMNSK (SEQ ID NO:1), AMESINSR (SEQ ID NO:2), and IDSTCGNVK (SEQ ID NO:3), respectively. The other peptide marker candidates failed to be identified. This may be caused by the limited protein information of EBN in the database. As searched in the UniprotKB, there were only 4,025 protein items for Apodidae and 104 protein items for Aerodramus fuciphagus (edible-nest swiftlet).

Table 3 summarizes the information of specific peptide markers of EBN, white EBN, and grass EBN.

| No. | Name | Observed m/z | RT (min) | Identified sample |
|---|---|---|---|---|
| 1 | BNM201 | 277.6012-277.7012 | 11.49 | White EBN |
| 2 | BNM202 | 280.1454-280.2454 | 12.86 | White EBN |
| 3 | BNM203 | 280.6246-280.7246 | 7.97 | EBN |
| 4 | BNM204 | 292.1148-292.2148 | 10.05 | White EBN |
| 5 | BNM205 | 294.7642-294.8642 | 20.7 | White EBN |
| 6 | BNM206 | 300.0959-300.1959 | 17.25 | EBN |
| 7 | BNM207 | 305.1166-305.2166 | 10.99 | White EBN |
| 8 | BNM208 | 321.6531-321.7531 | 20.45 | White EBN |
| 9 | BNM209 | 373.7747-373.8747 | 9.82 | EBN |
| 10 | BNM210 | 381.6542-381.7542 | 9.61 | EBN |
| 11 | BNM211 | 391.6929-391.7929 | 27.35 | White EBN |
| 12 | BNM212*/** | 404.1486-404.2486 | 16.65 | EBN |
| 13 | BNM213 | 410.6669-410.7669 | 27.35 | White EBN |
| 14 | BNM214 | 433.6506-433.7506 | 28.04 | White EBN |
| 15 | BNM215 | 441.6666-441.7666 | 20.65 | White EBN |
| 16 | BNM216*/** | 454.6518-454.7518 | 10.24 | White EBN |
| 17 | BNM217* | 468.6939-468.7939 | 20.78 | White EBN |
| 18 | BNM218 | 477.6830-477.7830 | 23.14 | EBN |
| 19 | BNM219 | 498.7467-498.8467 | 17.87 | EBN |
| 20 | BNM220 | 630.7600-630.8600 | 24.56 | EBN |
| 21 | BNM221 | 711.3910-711.3910 | 26.43 | White EBN |
| 22 | BNM222 | 820.3134-820.4134 | 15.13 | EBN |
| 23 | BNM223 | 844.3228-844.4228 | 20.63 | EBN |
| 24 | BNM224** | 335.1730-335.2730 | 15.39 | Grass EBN |
| 25 | BNM225 | 417.6510-417.7510 | 16.15 | Grass EBN |
| 26 | BNM226 | 447.6496-447.7496 | 9.81 | Grass EBN |

Note: The peptide markers marked as“*” are peptides which have been identified by searching the database. The peptide markers marked as “**” peptide markers were used for identification of EBN, white EBN and grass EBN identification in products.

Table 4 shows information of the identified peptide markers by searching database.

| | | | | | Pre- |
|---|---|---|---|---|---|
| | | | Ob- | | cur- |
| Peptide | | Protein | served | Charge | sor |
| Marker | Sequence | accessions | m/z | state | mass |
| BNM212 | NTLMNSK | tr\|A0A183VZ31\| | 404. | 2 | 806. |
| | (SEQ ID | A0A183VZ31_ | 2034 | | 4068 |
| | NO: 1) | TRIRE | | | |
| BNM216 | AMESINSR | tr\|B5M6F8\| | 454. | 2 | 907. |
| | (SEQ ID | B5M6 | 6704 | | 3408 |
| | NO: 2) | F8_CYRSC | | | |
| BNM217 | IDSTCGNVK | tr\|A0A183WAA0\| | 468. | 2 | 935. |
| | (SEQ ID | A0A183WAA0_ | 7120 | | 4240 |
| | NO: 3) | TRIRE | | | |

Peptide Marker detection in products: Using the three maker peptides (BNM 212, BNM216, BNM224), the EBN identification was applied to 46 batches of products collected from Hong Kong, Mainland China, and Vietnam. The retention time and mass data were used to confirm the existence of the three peptide markers. The result is shown in Table 5. The results show that about 21 batches had no EBN related peptide markers. The findings indicate that these products may have no or very low amount of EBN in these alleged EBN products, which suggests that development of an effective EBN authentication method would be quite meaningful to both customers and the industry. At the same time, 11 batches of products have both white and grass EBN peptide markers. And 14 batches products were found to be made from only white EBN. The results indicated that the incorrect labeling of white and grass nest, especially in EBN instant products, was very wide spread in the products tested.

Table 5 is the result of EBN, white EBN, and grass EBN identification in related products.

| Sample | | | |
|---|---|---|---|
| code | BNM212 | BNM216 | BNM224 |
| PD-01 | + | + | + |
| PD-02 | + | + | + |
| PD-03 | + | + | + |
| PD-04 | + | + | − |
| PD-05 | + | + | − |
| PD-06 | + | + | + |
| PD-07 | + | + | + |
| PD-08 | + | + | − |
| PD-09 | + | + | − |
| PD-10 | + | + | + |
| PD-11 | + | + | + |
| PD-12 | − | − | − |
| PD-13 | − | − | − |
| PD-14 | − | − | − |
| PD-15 | − | − | − |
| PD-16 | − | − | − |
| PD-17 | − | − | − |
| PD-18 | − | − | − |
| PD-19 | + | + | − |
| PD-20 | − | − | − |
| PD-21 | − | − | − |
| PD-22 | − | − | − |
| PD-23 | − | − | − |
| PD-24 | − | − | − |
| PD-25 | − | − | − |
| PD-26 | − | − | − |
| PD-27 | − | − | − |
| PD-28 | − | − | − |
| PD-29 | − | − | − |
| PD-30 | − | − | − |
| PD-31 | − | − | − |
| PD-32 | + | + | − |
| PD-33 | − | − | − |
| PD-34 | + | + | + |
| PD-35 | + | + | − |
| PD-36 | + | + | − |
| PD-37 | + | + | + |
| PD-38 | + | + | − |
| PD-39 | + | + | + |
| PD-40 | + | + | + |
| PD-41 | + | + | − |
| PD-42 | − | − | − |
| PD-43 | + | + | − |
| PD-44 | + | + | − |
| PD-45 | + | + | − |
| PD-46 | + | + | − |

Note:
+ the peptide marker could be detected at the same retention time;
− the peptide marker fails to be detected at the same retention time.

In summary, the present disclosure relates to the peptide markers. These peptide markers were from hydrolysis, such as by tryptic digestion, of EBN. These peptide markers could be used to identify EBN, distinguish white EBN and grass EBN no matter what the EBN is pure or present in a mixture with adulterants. The detection of these peptide markers is not limited to LC-ESI-qTOE-MS/MS analysis. Any strategy could be employed if applicable. Compared with current methods, the method described herein is specific, simple and commercial application friendly.

### Experimental

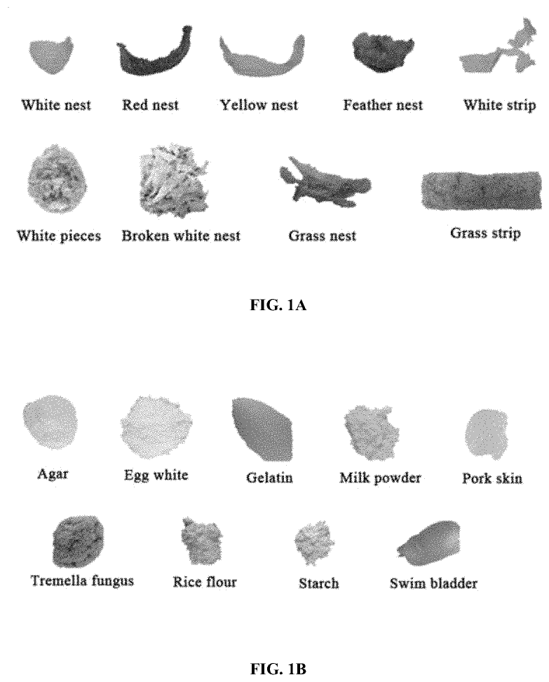

Materials and Chemicals: The multiple batches of EBN with sample number (n) equals to 70 includes white EBN (n=60) and grass EBN (n=10) were collected from local market (Hong Kong, China). The agar (n=10), egg white (n=5), gelatin (n=8), cow milk (n=8), pork skin (n=4), tremella fungus (n=6), rice flour (n=4), starch (n=3) and swim bladder (n=3) were purchased from the market (Hong Kong, China). The typical morphology was shown in FIGS. 1A and 1B. These samples were fully dried and then grinded into powder. The EBN related products (n=46) were purchased from the market. The trypsin was obtained in the National Institutes for Food and Drug Control (Beijing, China). The sodium bicarbonate and other related chemical regents were all purchased from Sigma.

Enzymatic digestion: The powder (5 mg) of the EBN sample or adulterants was dissolved in 500 μL1% ammonium bicarbonate (ABC) and sonicated for 30 min. For EBN products, 250 μL product was diluted with 250 μL 2% ammonium bicarbonate (ABC). Then 50 μL trypsin (10 mg/mL in 1% ABC) with the weight ratio of 1:10 (trypsin: sample=1:10) was added. The mixture was incubated at 37° C. for 18 h. After digestion, the hydrolysate solution was acidified with 20 μL formic acid to pH≤4. Then 900 μL 1% ABC was added to dilute the samples. After that, 400 μL of the diluted sample was mixed with 1 mL methanol. The mixture was centrifugated at 15,000 rpm for 15 min. The supernatant was collected for further LC-ESI-Q-TOF-MS/MS analysis.

LC-qTOE-MS/MS conditions: The separation was performed on an Agilent 1290 UHPLC system (Agilent Technologies, Santa Clara, USA) equipped with a binary pump, a thermostatic column compartment, an auto-sampler, and a degasser and a diode-array detector. The system was controlled by Mass Hunter B.06 software. The chromatographic column ACQUITY UPLC BEH C18 (2.1 mm×100 mm, 1.7 μm, Waters, Milford, USA) was used and eluted with a linear gradient of 0.1% formic acid in water (A) and 0.1% formic acid in acetonitrile (B) at a flow rate of 0.35 mL/min and at a temperature of 40° C. The solvent gradient programming was as follows: 0-5 min, 1% B; 5-30 min, 1-25% B; 30-32 min, 25-75% B; 32-33 min, 75-100% B; 33-34.1 min, 100-1% B; 34.1-38 min, 1% B. The injection volume was 2 μL.

MS data were collected form an Agilent 6540 Q-TOF mass spectrometer (Agilent Technologies, Santa Clara, USA) equipped with quadrupole-time-of-flight (Q-TOF) mass spectrometer and a JetStream electrospray ion (ESI) source. Data acquisition was controlled by MassHunter B.06 software (Agilent Technologies). The optimized operating parameters in the negative ion mode were as follows: nebulizing gas (N₂) flow rate, 7.0 L/min; nebulizing gas temperature, 300° C.; Jet Stream gas flow, 8 L/min; sheath gas temperature, 350° C.; nebulizer, 40 psi; capillary, 3000 V; skimmer, 65 V; Oct RFV, 600 V; and fragmentor voltage, 150 V. The mass scan range was set as mass to charge (m/z) 100-2000. MS/MS technique could provide parallel alternating scans for acquisition at low collision energy to obtain precursor ion information or at a ramping of high collision energy to obtain a full-scan accurate mass of fragments, precursor ions and neutral loss information. The collision energies for Auto MS/MS analysis were 20 V and 40 V, respectively.

Peptide marker selection for EBN: The peptide profiles of digested EBN and the adulterants were extracted ions from scan range of m/z 100-400, m/z 400-800 and m/z 800-1200 for further comparison. To simplify the selection procedures, three criteria should be followed. Firstly, the peptides with similar mass fragmentations and retention time in different scan range were considered as the same compounds. Secondly, the observed m/z with the charge state of 1 should not be a peptide marker candidate. Thirdly, the peptide selected should be only found in multiple digested EBN other than any of the adulterants.

Peptide marker selection for distinguishing white and grass EBN: For the white and grass EBN differentiation, the criteria is the same as the peptide marker selection for EBN. At the same time, the peptide selected should be only found in multiple white EBN or grass EBN.

Database searching: After QTOF-MS/MS analysis, the SGF files were loaded into ProteinPilot 5.0 software to match proteins and peptides. The corresponding data were searched against Ayes (1,035,750 protein items) databases downloaded from UniProt KB. The parameters of ProteinPilot 5.0 were: Trypsin digestion and Rapid search effort. An integrated false discovery rate (FDR) analysis was applied to establish a confidence level for the results.

### INDUSTRIAL APPLICABILITY

The present invention discloses a series of peptide markers. These peptides were from the EBN after trypsin digestion. Using these peptide markers, it provides a simple and low-cost, high sensitivity and specificity, repeatable and practical for commercial application in the market. This invention provides a method to authentic EBN and related products. Simultaneously, this invention also provides a method to identify white EBN, grass EBN in raw material and related products by using these markers.

What is claimed:

1. A method of identifying an edible bird's nest (EBN) in a sample suspected of comprising the EBN, the method comprising:
providing a hydrolysate of the sample;
analyzing the hydrolysate using a mass spectroscopy method;
determining whether the hydrolysate comprises one or more peptide markers, wherein the one or more peptide markers have an observed mass to charge ratio (m/z) selected from the group consisting of: 277.6012-277.7012 (BNM201), 280.1454-280.2454 (BNM202), 280.6246-280.7246 (BNM203), 292.1148-292.2148 (BNM204), 294.7642-294.8642 (BNM205), 300.0959-300.1959 (BNM206), 305.1166-305.2166 (BNM207), 321.6531-321.7531 (BNM208), 373.7747-373.8747 (BNM209), 381.6542-381.7542 (BNM210), 391.6929-391.7929 (BNM211), 404.1486-404.2486 (BNM212), 410.6669-410.7669 (BNM213), 433.6506-433.7506 (BNM214), 441.6666-441.7666 (BNM215), 454.6518-454.7518 (BNM216), 468.6939-468.7939 (BNM217), 477.6830-477.7830 (BNM218), 498.7467-498.8467 (BNM219), 630.7600-630.8600 (BNM220), 711.3910-711.3910 (BNM221), 820.3134-820.4134 (BNM222), 844.3228-844.4228 (BNM223), 335.1730-335.2730 (BNM224), 417.6510-417.7510 (BNM225), and 447.6496-447.7496 (BNM226); and
identifying based on the whether the hydrolysate comprises the one or more peptide markers if the sample comprises the EBN.

2. The method of claim 1 further comprising the step of hydrolyzing the sample thereby forming the hydrolysate of the sample.

3. The method of claim 1 further comprising the step of hydrolyzing the sample using a protease thereby forming the hydrolysate of the sample.

4. The method of claim 3, wherein the protease is selected from the group consisting of trypsin, chymotrypsin, lysine protease, aspartic protease, pepsin, papain, proteinase K, calpain, and subtilisin.

5. The method of claim 3, wherein the protease is trypsin.

6. The method of claim 1, wherein the mass spectrometry method is tandem mass spectroscopy (MS/MS) and further comprises a liquid chromatography method

7. The method of claim 1, wherein the mass spectrometry method comprises high-performance liquid chromatography (HPLC-MS/MS) or ultra-performance liquid chromatography (UPLC-MS/MS).

8. The method of claim 6, wherein the step of determining whether the hydrolysate comprises one or more peptide markers further comprises determining whether each of the one or more peptide markers has a predicted liquid chromatography retention time, wherein the predicted liquid chromatography retention time is determined by measuring the retention time of standard samples, wherein each of the standard samples comprises one of the one or more peptide markers.

9. The method of claim 1, wherein the one or more peptide markers having an observed m/z are selected from the group consisting of BNM212, BNM216, and BNM224.

10. The method of claim 9, wherein the step of determining whether the hydrolysate comprises one or more peptide markers comprises determining whether the hydrolysate comprises 2 or 3 peptide markers selected from the group consisting of BNM212, BNM216, and BNM224.

11. The method of claim 9, wherein the step of determining whether the hydrolysate comprises one or more peptide markers comprises determining whether the hydrolysate comprises 3 peptide markers selected from the group consisting of BNM212, BNM216, and BNM224.

12. The method of claim 1, wherein the step of determining whether the hydrolysate comprises one or more peptide markers comprises determining whether the hydrolysate comprises 10 or more peptide markers, 15 or more peptide markers, 20 or more peptide markers, or 23 or more peptide markers.

13. The method of claim 1, wherein the step of determining whether the hydrolysate comprises one or more peptide markers comprises determining whether the hydrolysate comprises 23 peptide markers selected from the group consisting of BNM201, BNM202, BNM203, BNM204, BNM205, BNM206, BNM207, BNM208, BNM209, BNM210, BNM211, BNM212, BNM213, BNM214, BNM215, BNM216, BNM217, BNM218, BNM219, BNM220, BNM221, BNM222, and BNM223; and optionally three peptide markers from the group consisting of BNM224, BNM225 and BNM226; and the step of identifying based on the whether the hydrolysate comprises the one or more peptide markers if the sample comprises EBN optionally comprises identifying if the sample comprises EBN, white EBN, or grass EBN.

14. The method of claim 1, wherein the one or more peptide markers having an observed m/z are selected from the group consisting of BNM201, BNM202, BNM204, BNM205, BNM207, BNM208, BNM211, BNM213, BNM214, BNM215, BNM216, BNM217 and BNM221; and the step of identifying based on the whether the hydrolysate comprises the one or more peptide markers if the sample comprises EBN optionally comprises identifying if the sample comprises white EBN.

15. The method of claim 14, wherein the step of determining whether the hydrolysate comprises one or more peptide markers comprises determining whether the hydrolysate comprises 2 or more peptide markers, 3 or more peptide markers, 4 or more peptide markers, 5 or more peptide markers, 6 or more peptide markers, 7 or more peptide markers, 8 or more peptide markers, 9 or more peptide markers, 10 or more peptide markers, 11 or more peptide markers, or 12 or more peptide markers from the group consisting of BNM201, BNM202, BNM204, BNM205, BNM207, BNM208, BNM211, BNM213, BNM214, BNM215, BNM216, BNM217 and BNM221.

16. The method of claim 14, wherein the step of determining whether the hydrolysate comprises one or more peptide markers comprises determining whether the hydrolysate comprises thirteen peptide markers from the group consisting of BNM201, BNM202, BNM204, BNM205, BNM207, BNM208, BNM211, BNM213, BNM214, BNM215, BNM216, BNM217 and BNM221.

17. The method of claim 1, wherein the one or more peptide markers having an observed m/z are selected from the group consisting of BNM224, BNM225 and BNM226; and the step of identifying based on the whether the hydrolysate comprises the one or more peptide markers if the sample comprises EBN optionally comprises identifying if the sample comprises grass EBN.

18. The method of claim 14, wherein the step of determining whether the hydrolysate comprises one or more peptide markers comprises determining whether the hydrolysate comprises 2 or 3 peptide markers selected from the group consisting of BNM224, BNM225 and BNM226.

19. The method of claim 1, wherein the method comprises:
providing a trypsin hydrolysate of the sample;
analyzing the trypsin hydrolysate using an UPLC-MS/MS method;
determining whether the trypsin hydrolysate comprises peptide markers selected from the group consisting of:
3 peptide markers having an observed m/z are selected from the group consisting of BNM212, BNM216, and BNM224;
23 peptide markers having an observed m/z are selected from the group consisting of BNM201, BNM202, BNM203, BNM204, BNM205, BNM206, BNM207, BNM208, BNM209, BNM210, BNM211, BNM212, BNM213, BNM214, BNM215, BNM216, BNM217, BNM218, BNM219, BNM220, BNM221, BNM222, and BNM223;
13 peptide markers having an observed m/z are selected from the group consisting of BNM201, BNM202, BNM204, BNM205, BNM207, BNM208, BNM211, BNM213, BNM214, BNM215, BNM216, BNM217 and BNM221; and
3 peptide markers having an observed m/z are selected from the group consisting of BNM224, BNM225, and BNM226; and
identifying based on the whether the trypsin hydrolysate comprises the peptide markers if the sample comprises EBN, white EBN, or grass EBN.

20. The method of claim 19, wherein the step of determining whether the hydrolysate comprises one or more peptide markers further comprises determining whether each of the one or more peptide markers has a predicted ultra-performance liquid chromatography retention time, wherein the ultra-performance predicted liquid chromatography retention time is determined by measuring the retention time of standard samples, wherein each of the standard samples comprises one of the one or more peptide markers.


SEQUENCE LISTING (3 sequences)

SEQ ID NO: 1 - protein, 7 aa, Artificial Sequence
  Artificial sequence prepared in the lab
NTLMNSK

SEQ ID NO: 2 - protein, 8 aa, Artificial Sequence
  Artificial sequence prepared in the lab
AMESINSR

SEQ ID NO: 3 - protein, 9 aa, Artificial Sequence
  Artificial sequence prepared in the lab
IDSTCGNVK